(19) United States
(12) Patent Application Publication
(10) Pub. No.: US 2007/0293708 A1
(43) Pub. Date: Dec. 20, 2007

(54) Process for preparing 2-bromo-4,4'-dialkylbiphenyl

(75) Inventor: Florian Rampf, Hegenheim (FR)

Correspondence Address: LANXESS CORPORATION, 111 RIDC PARK WEST DRIVE, PITTSBURGH, PA, 15275-1112, US

(73) Assignee: Saltigo GmbH

(21) Appl. No.: 11/811,080

(22) Filed: Jun. 8, 2007

(30) Foreign Application Priority Data
Jun. 14, 2006 (DE) 10 2006 027 491.1

(51) Int. Cl. C07C 19/00

(52) U.S. Cl. 570/253

(57) ABSTRACT

Process for preparing 2-bromo-4,4′-dialkylbiphenyl, wherein 4,4′-dialkylbiphenyl is, in the presence of catalytic amounts of iodine and, if appropriate, in a halogenated hydrocarbon as solvent, reacted with bromine.

The present invention relates to a process for preparing 2-bromo-4,4′-dialkylbiphenyl, in particular 2-bromo-4,4′-di-tert-butylphenyl. These bromine compounds play an important role in the synthesis of ligands for C—C couplings; they can be used, for example, to construct the skeleton of ligands for the Buchwald reaction.

J.Org. Chem., 1979, 44 (17), 3037-3041 describes the bromination of 4,4′-di-tert-butylbiphenyl with bromine in the presence of iron as catalyst in a yield of 73%. Here, biphenyl is initially converted into 4,4′-di-tert-butylbiphenyl. This compound is then brominated, with the tert-butyl group serving as a directing protective group. This protective group is then removed, liberating the product. It was found that this synthesis, where the catalyst used is iron, does not provide the desired bromination selectivity and thus yield. In addition to the desired monobrominated product, isomeric monobrominated and dibrominated products and also products where one or both tert-butyl groups are lost are formed. Accordingly, it is difficult to isolate the desired product 2-bromo-4,4′-di-tert-butylbiphenyl from the reaction mixture. In addition, the solvent used for the bromination is carbon tetrachloride, which causes problems when the reaction is carried out on an industrial scale.

Accordingly, it was an object of the present invention to provide a process for preparing 2-bromo-4,4′-dialkylbiphenyl by brominating the corresponding 4,4′-dialkylbiphenyl compound with virtually complete selectivity and very good yield. Surprisingly, it has now been found that this reaction takes place with virtually complete selectivity and very good yield if the bromination is carried out in the presence of catalytic amounts of iodine (I₂).

Accordingly, the invention provides a process for preparing 2-bromo-4,4′-dialkylbiphenyl which comprises reacting 4,4′-dialkylbiphenyl with bromine in the presence of catalytic amounts of iodine and, if appropriate, in a halogenated hydrocarbon as solvent.

Suitable 4,4′-alkylbipheny compounds are the corresponding methyl, ethyl, propyl and butyl compounds. Preference is given here to 4,4′-di-tert-butylbiphenyl, where the bromination affords the desired product 2-bromo-4,4′-di-tert-butylbiphenyl. Here, the iodine is present as elemental iodine in a catalytic amount, for example in an amount of from 0.1 to 10 mol %, based on the aromatic compound to be brominated. Preferably, the amount of iodine used is from 1 to 5 mol %, based on the aromatic compound to be brominated.

The reaction can be carried out in the absence of a solvent, but is preferably carried out in a nonaqueous solvent, such as a halogenated hydrocarbon. Suitable for use as halogenated hydrocarbons are all liquid hydrocarbon compounds from the group consisting of methylene chloride, 1,2-dichloroethane, chloroform, chlorobenzene and dichlorobenzene. Preferably, the solvent used is methylene chloride.

The reaction is usually carried out at a temperature in the range of from −5 to 40° C. The preferred reaction temperature is 5° C.

The reaction time for the reaction according to the invention is in the range between one and 10 hours. Preferably, the reaction time is from one to two hours. Surprisingly, using the process according to the invention, it is possible to obtain the bromobiphenyl compounds mentioned in high selectivity and yield. The selectivity is usually at more than 98%, and the yield at more than 95%.

The example below is meant to illustrate the invention in more detail, but without limiting its scope.

### EXAMPLE 1

1 g of 4,4′-di-tert-butylbiphenyl were dissolved in 10 g of dichloromethane, and 30 mg of iodine were added. The mixture was then cooled to 5° C. Over a period of 1 h, 650 mg of bromine were added. The mixture was then stirred at room temperature for 1 h. For work-up, 3 ml of a 10% strength solution of NaHSO₃ were added, the phases were separated and the organic phase was washed until neutral, dried and concentrated using a rotary evaporator. This gave 1.28 g of 2-bromo-4,4′-di-tert-butylbiphenyl (98.6% of theory).

1. Process for preparing 2-bromo-4,4′-dialkylbiphenyl, wherein 4,4′-dialkylbiphenyl is, in the presence of catalytic amounts of iodine and, if appropriate, in a halogenated hydrocarbon as solvent, reacted with bromine.

2. Process according to claim 1, wherein the 2-bromo-4,4′-dialkylbiphenyl is 2-bromo-4,4′-di-tert-butylbiphenyl.

3. Process according to claim 1 or 2, wherein the iodine is employed in an amount of from 0.1-10 mol %, based on the 4,4′-dialkylbiphenyl.

4. Process according to any of the preceding claims, wherein the halogenated hydrocarbon is methylene chloride.

5. Process according to any of the preceding claims, wherein the reaction is carried out at a temperature in the range of 0-40° C.

6. Process according to any of the preceding claims, wherein the reaction time is between one and 10 h.
